(19) United States
(12) Patent Application Publication
(10) Pub. No.: US 2023/0288408 A1
(43) Pub. Date: Sep. 14, 2023

(54) NOVEL PEPTIDE TAG

(71) Applicants: Technische Universität Müncher, München (DE); Karlsruher Institut für Technologie, Karlsruhe (DE)

(72) Inventors: Sonja Berensmeier, München (DE); Sebastian Schwaminger, Graz (AT); Silvia Blank-shim, Aschaffenburg (DE); Paula Fraga García, Freising (DE); Yasmin Kaveh-Baghbaderani, München (DE); Alexander Zanker, München (DE); Stefan Rauwolf, München (DE); Wolfgang Wenzel, Bruchsal (DE)

(21) Appl. No.: 18/015,906

(22) PCT Filed: Jul. 15, 2021
(86) PCT No.: PCT/EP2021/069795

§ 371 Date: Jan. 12, 2023

(30) Foreign Application Priority Data
Jul. 15, 2020 (EP) 20185909.7

(51) Int. Cl. G01N 33/543; C07K 7/06

(52) U.S. Cl. CPC G01N 33/54326; C07K 7/06; C07K 2319/20

(57) ABSTRACT

The present invention relates to a peptide consisting of a sequence of 5 or 6 to 30, preferably 6 to 12, most preferably 10 to 12 amino acids, wherein (a) at least ⅓, preferably at least ½ of said amino acids are amino acids having a functional group or a side chain which is positively charged at neutral pH; and (b) at least two amino acids, preferably at least three are histidines.

The present invention relates to a peptide consisting of a sequence of 5 or 6 to 30, preferably 6 to 12, most preferably 10 to 12 amino acids, wherein (a) at least ⅓, preferably at least ½ of said amino acids are amino acids having a functional group or a side chain which is positively charged at neutral pH; and (b) at least two amino acids, preferably at least three are histidines.

In this specification, a number of documents including patent applications and manufacturer’s manuals are cited. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

In biotechnology, interfacial interactions play important roles for multiple purification processes, for enzyme immobilization and for biosensor applications. Therefore, it is of great interest to understand these interactions and exploit them. Here, distinct peptide sequences come into play, which specifically bind inorganic surfaces. Such short peptide sequences consisting of up to 30 amino acids are called (binding or recognition) tags. These tags facilitate a specific binding to a surface and allow for purification or immobilization of proteins fused to such sequences. Typical examples for such peptide sequences are the His-tag, Flag-tag, Glu-tag or Strep-tag.^{1,2} The purification of proteins from complex mixtures such as crude cell lysates is typically conducted with affinity chromatography via a directed binding to the respective recognition site immobilized on the chromatography resin. Usually, complexation, ionic and hydrophobic interactions, or mixtures of these interactions between the recognition site and the tag can be applied in protein purification and directed protein immobilization.

Iron oxide nanoparticles (IONs) are widely used for the immobilization of proteins and other biological molecules.^{3,4} Other applications of IONs include drug delivery, hyperthermia treatment and, use as contrast agents for magnetic resonance imaging.⁵ To date, iron oxide nanomaterials are mostly used in the form of magnetic microbeads for analytical and biochemical purposes.⁶

Of particular interest are iron oxide nanoparticles (or microparticles) since they constitute low-cost material and it is simple to process them. They can be used, for example, as an alternative to chromatography resin. Magnetic iron oxide nanomaterials are usually based on magnetite (Fe₃O₄), maghemite (γ-Fe₂O₃) or transition states in between those two materials.⁷ These materials show a high biocompatibility, a high saturation magnetization (> 80 Am²/kg) and superparamagnetic behavior and thus no remanence (< 0.5 Am²/kg). There are multiple synthesis strategies to generate iron oxide nanoparticles but the Massart process depicts the most simple and cost-efficient route.^{8,9}

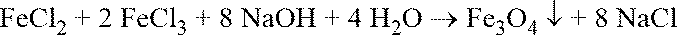

To date only few publications directly address the binding of peptides to bare iron oxide or silica surfaces.^{1,10,11} Therefore, such surfaces are commonly functionalized with ligands and polymers for chromatography and magnetic separation processes which show a high selectivity compared to “classical” tags.¹² In order to be used in a number of applications, IONs can be functionalized to selectively bind to their respective interaction partners. This can presently be achieved by attaching metal ion chelating molecules like nitrilotriacetic acid to the ION surface so that His-tagged biomolecules can be bound.¹³ Drawbacks of this method are the leakage of toxic metal ions and instability of the surface functionalization.^{14,15} Other surface modifications for protein adsorption on magnetic particles use glutathione, streptavidin, biotin or protein A, but all of these lead to high costs of over 400 to several thousand euros per gram.¹⁶ Problems of most covalent immobilization techniques such as amino-silanization are the high complexity and loss of enzymatic activity due to the reaction of catalytic amino acids.^{17,18}

The functionalization is a cost-intensive process and avoiding such an additional step would directly result in economic benefits. For these reasons, it would be desirable and advantageous to overcome the requirement of application-specific functionalization and to engineer tags such as peptide sequences that can bind to IONs. In case of the tags being peptides, they can directly be encoded by those nucleic acids which also encode the polypeptide of interest for a given application.

In view of the discussed deficiencies of the prior art, the technical problem underlying the present invention can be seen in the provision of improved means and methods of binding molecules such as biomolecules or analytes in general to surfaces such as iron oxide-containing surfaces or silica-containing surfaces, especially magnetic particles. This technical problem is solved by the present invention.

Hence, the present invention relates in a first aspect to a peptide consisting of a sequence of 5 or 6 to 30, preferably 6 to 12, most preferably 10 to 12 amino acids, wherein (a) at least ⅓, preferably at least ½ of said amino acids are amino acids having a functional group or a side chain which is positively charged at neutral pH; and (b) at least two amino acids, preferably at least three are histidines.

The term “peptide” has its art-established meaning. It relates to a polycondensate of amino acids, the number of amino acids being 30 or less. In accordance with the invention, peptides have length between 5 and 30 amino acids. Envisaged lengths are 5, 6, 7, 8, 9, 10, 11, 12, 13, 14, 15, 16, 17, 18, 19, 20, 21, 22, 23, 24, 25, 26, 27, 28, 29 and 30 amino acids. The range of the length of the peptides may be, for example, 6 to 30, 8 to 30, 9 to 30, 10 to 30, 11 to 30, 12 to 30, 13 to 30, 14 to 30, 15 to 30, 16 to 30, 17 to 30, 18 to 30, 19 to 30, 20 to 30, 21 to 30, 22 to 30, 23 to 30, 24 to 30, 25 to 30, 26 to 30, 27 to 30, 28 to 30, 29 or 30 amino acids, or the length of the peptides may be 30 amino acids. Peptides may be chemically synthesized or translated from nucleic acids encoding them. The term “peptide” as used herein does not imply a limitation to α-amino acids. Therefore, the charge status of the amino acids is a feature of a functional group comprised in a given amino acid.

To the extent the amino acids are α-amino acids, the term “side chain” has its usual meaning and designates group R in the following generic formula: —NH—CHR—CO—. The side chain may carry a functional group which may be charged at neutral pH and which in accordance with item (a) of the first aspect is positively charged at neutral pH.

Also, a “functional group” has its usual meaning and designates specific substituents or moieties within molecules that are responsible for the characteristic chemical reactions of those molecules. Non-limiting examples of functional groups that may be positively charged at neutral pH (and thus may accept a proton at neutral pH) are —NH₂ and ═NH, and non-limiting examples of functional groups that may be negatively charged at neutral pH (and thus may donate a proton at neutral pH) are —COOH, —HSO₄, and —H₂PO₄.

Whether or not a given amino acid is positively or negatively charged at neutral pH may be determined by assessing an aqueous solution of the amino acid at issue in its monomeric form, i.e., with free amino and carboxy terminus. Alternatively, the N-terminus and/or the C-terminus may be protected, for example, the N-terminus may be acetylated, and the C-terminus may be amidated. pKa value(s) of an amino acid can be determined by acid/base titration. The pKa value in turn determines the charge status at a given pH value.

Amino acids are preferably α-amino acids, wherein the naturally occurring 20 proteinogenic amino acids are especially preferred. To the extent use is made of proteinogenic amino acids, peptides of the present invention as well as fusion constructs, namely polypeptides and proteins as disclosed further below can be encoded by nucleic acids. Having said that, it is also envisaged to use other amino acids, wherein said other amino acids include α-amino acids which do not belong to the set of 20 proteinogenic amino acids such as pyrrolysine and selenocysteine. Among α-amino acids, preference is given to L-amino acids. At the same time, the use of D-amino acids is also envisaged. Moreover, one or more amino acids which are not α-amino acids, such as β-amino acids may be present. Preference is given to absence of non-proteinogenic amino acids. Yet, any of the above-mentioned non-proteinogenic amino acids may be present, preferably in small numbers such as 1, 2, 3, 4 or 5. It is not excluded that also 6, 7, 8, 9 or 10 non-proteinogenic amino acids are comprised in peptides in accordance with the first aspect of the invention.

In accordance with item (a) of the first aspect, at least ⅓, preferably at least ½ of said amino acids are amino acids having a functional group or a side chain which is positively charged at neutral pH. It is understood that at least in a preferred embodiment said amino acids do not contain a functional group or side chain which is negatively charged at neutral pH.

A neutral pH is a pH of about 7.0, wherein the term “about” is ±10%, preferably ±5%. Among the proteinogenic amino acids, this requirement is fulfilled by lysine (isoelectric point 9.74) and arginine (isoelectric point 10.76). Accordingly, an amino acid having a functional group or a side chain which is positively charged at neutral pH is preferably an amino acid having an isoelectric point of at least 9.0, preferably of at least 9.5.

For instance, if a peptide consists of 9 amino acids and at least ⅓ of said 9 amino acids are amino acids having a functional group or a side chain which is positively charged at neutral pH this means that in accordance with requirement (a), 3, 4, 5, 6 or 7 amino acids have a functional group or a side chain which is positively charged at neutral pH, noting in accordance with requirement (b) at least two amino acids are histidines.

In accordance with item (b) of the first aspect, at least two amino acids, preferably at least three amino acids are histidines. Histidine contains an α-amino group (which is in the protonated -NH₃⁺ form under biological conditions), a carboxylic acid group (which is in the deprotonated -COO⁻ form under biological conditions), and an imidazole side chain (which is partially protonated). Although histidine is classified as a positively charged amino acid it has an isoelectric point of 7.59. This is because the imidazole side chain of histidine is only partially protonated at neutral pH. It follows that in accordance with the present invention histidine is not an amino acid having a functional group or a side chain which is positively charged at neutral pH.

As can be taken from the appended examples it was found in connection with the present invention that peptide sequences containing arginine/lysine and histidine bind extremely well to iron oxide surfaces. Peptides containing arginine/lysine and histidine sequences are also very good silica binders. It was furthermore found that the binding to iron oxide surfaces is irreversible, whereas the binding to silica is reversible. The peptide sequences can be separated from the silica surface, for example, with a lysine or arginine buffer (usually > 100 mM).

The surprising findings as illustrated by the appended examples may be summarized as:

1) Peptides containing a high arginine/lysine content and histidine display better binding to iron oxide and silica nanoparticles than peptides with a high content of arginine. The peptides containing arginine/lysine and histidine bind with a higher affinity to iron oxide surfaces than other peptides and, thus, inter alia facilitate their direct immobilization from complex mixtures, such as cell lysates.

2) The binding to iron oxide surfaces is irreversible and the addition of further solutes does not break the bond between the peptide tag and the iron oxide surface.

3) The binding to silica surfaces is reversible and, for example, the addition of a lysine or arginine buffer breaks the bond between the peptides and the surface.

Hence, the peptide of the invention is capable of binding to a surface comprising iron oxide or silica, preferably a magnetic nanoparticle or a porous silica particle.

As will be further detailed herein below, nucleic acid sequences inter alia encoding peptides of the invention can be integrated into an expression vector or the genome of a microorganism, which further allows for the expression of a target protein with the attached peptide tag. Tagged proteins may bind to iron oxide surfaces through the peptide tag of the invention. The peptides can also be used to immobilize sensor/recognition proteins (such as antibodies or enzymes). The peptides can furthermore be fused by chemical processes to target biomolecules. Also, binding can be simulated since only the binding protein tag sequences bind to surfaces.

Furthermore, the extremely efficient antibody purification by immobilized protein A-domains, which are immobilized on iron oxide nanoparticles via the peptide tag of the invention is highlighted (Example 3). This application of the peptide tag of the invention and the discussed application as a novel complex of proteins as domains being immobilized on iron oxide nanomaterials (Example 2) have a high commercial value. Finally, the peptide tag of the invention can be employed in purification methods by classical IMAC (immobilized metal ion affinity chromatography) processes.

Hence, the peptides of the invention provide for a plethora of possibilities to use them in practical applications which applications are technically improved practical applications as compared to related art-established practical applications. As discussed herein above, the functionalization of iron oxide and silica surfaces, such as nanoparticles as required by related art-established practical applications is a cost-intensive process and the use of the peptide tags of the invention makes the functionalization unnecessary. The peptide tags of the invention sequences can directly bind to iron oxide and silica surfaces, and the tags being peptides, can directly be encoded by nucleic acids which also encode a polypeptide or protein of interest for a given application.

The peptides of the invention therefore render the art-established application-specific functionalization of magnetic nanoparticles and silica particles dispensable. The art-established functionalization of the surface of nanoparticles/porous micro particles does not only entail the need for a specific modification of the particles for any given specific application, it also is sometimes instable or leaks. Hence, the present invention as disclosed herein saves time and costs.

In accordance with a preferred embodiment of the first aspect the amino acids having a functional group or a side chain which is positively charged at neutral pH are lysines, arginines or a mixture of lysine(s) and arginine(s).

As discussed herein above, among the proteinogenic amino acids lysine and arginine are the amino acids having a functional group or a side chain which is positively charged at neutral pH. However, also a number of non-proteinogenic amino acids are amino acids having a functional group or a side chain which is positively charged at neutral pH. Non-limiting examples are homolysine and homoarginine.

In accordance with a further preferred embodiment of the first aspect the peptide comprises at least three amino acids having a functional group or a side chain which is positively charged at neutral pH.

As also discussed herein above, peptides with arginine/lysine and histidine have a surprisingly high binding affinity to metal oxide and silica surfaces. In particular, peptides with at least three arginines/lysines and at least two, preferably at least three histidines ensure that the peptide of the invention comprises a sufficient number of arginines/lysines and histidines in order to display a surprisingly high binding affinity to metal oxide and silica surfaces.

In accordance with a further preferred embodiment of the first aspect the peptide comprises at least one, preferably at least two and most preferably at least three alternating region(s) (S) and regions (W) each, wherein each region (S) comprises at least one amino acid having a functional group or a side chain which is positively charged at neutral pH, and each region (W) comprises at least one histidine.

This aspect of the invention relates to the secondary structure of peptides. The peptide folding is dependent on the side chains such as R, K and H. In order to be stabilized best, similar charges oppose each other, which means that such peptides are usually oriented as an alpha helix. If this structure is not broken by the binding process, only half of the side chains can be oriented to the surface. Here flexible or rigid regions can help to optimize the amount of positively charged side chains and histidine side chains which are oriented to the surface. Even a lower total number of R, K and H can therefore be preferential for this tag system.

To have histidines and lysines in different binding domains can have the advantage of improving the binding effect. Since two/three histidines in direct neighborhood bind better than just one, it makes sense to separate the binding of histidine and lysine to iron oxide surfaces since the coupling mechanism is different.

This aspect of the invention introduces the notion of regions within the sequence of the peptide. In particular, the designations (S) and (W) refer to groups of adjacent amino acids within the peptide of the invention which confer strong (S) binding to a surface comprising iron oxide or silica and groups which consist of adjacent amino acids which confer weak (W) binding to such surface, respectively.

Examples of alternating (S) and (W) regions include (S)(W), (W)(S), (S)(W)(S), (W)(S)(W), (S)(W)(S)(W) and (W)(S)(W)(S). A preferred pattern is (S)(W)(S). This applies to all aspects of the invention.

By the amino acids within the peptide of the invention which confer strong (S) certain characteristics, in particular the binding characteristics of the peptide tag of the invention can be set as needed. For instance, some amino acid units such as alanine and glycine add flexibility to the peptide tag, which can be beneficial for the binding if more positively charged groups can be brought to the surface due to the flexible tag. On the other hand, prolines can increase the stiffness of the linker, which can also be beneficial in order to draw the binding of positively charged peptides away from the protein (which might be negatively charged) and therefore direct/align the binding domains to the surface.

In a preferred embodiment of the first aspect the peptide is a “homopolymer” of amino acids which have a functional group or side chain which is positively charged at neutral pH and His which does not comprise any other amino acids. Particularly preferred is that the peptide is a homopolymer of Arg and/or Lys and His.

In the examples herein below the homopolymers (HR)₄ and (RH)₄ are used as peptide tags.

In accordance with a yet further preferred embodiment of the first aspect the at least two, preferably at least three histidines are present in a stretch or no more than 8 consecutive amino acids, preferably not more than 6 consecutive amino acids, and most preferably not more than 4 consecutive amino acids.

Hence, in accordance with this preferred embodiment the histidines are in close proximity within the peptide sequence. This in turn ensures that the histidines can optimally contribute together with the arginines and/or lysines to the high affinity binding to metal oxide or silica surfaces.

In accordance with another preferred embodiment of the first aspect any amino acids present in the peptide and having no functional group or side chain or having a functional group or side chain which is negatively charged at neutral pH are present in the peptide up to ⅕, preferably up to ⅒ and most preferably up to 1/20 of all amino acids of the peptide, and wherein said amino acids preferably (i) do have a functional group or a side chain which is negatively charged at neutral pH, and/or (ii) have a functional group or a side chain which does not bear a net charge at neutral pH.

As discussed, amino acids which have a side chain which is positively charged at neutral pH together with histidines ensure irreversible binding to iron oxide surfaces. Keeping the amino acids within the peptide which have no functional group or side chain or having a functional group or side chain which is negatively charged at neutral pH at a level of up to ⅕, preferably up to ⅒ and most preferably up to 1/20 of all amino acids of the peptide ensures that the irreversible binding is not disrupted or partially disrupted by negative charges.

Said amino acids within the peptide which have no functional group or side chain or have a functional group or side chain which is negatively charged at neutral pH, if present, are preferably each flanked on one side, preferably both sides by at least one, preferably at least two amino acids having a side chain which is positively charged at neutral pH or by histidine. This is because amino acids with side chains which do not bear a net charge at neutral pH contribute weakly to binding of said peptide to a surface comprising iron oxide and silica and amino acids having a functional group or side chain which is negatively charged at neutral pH do contribute to binding of said peptide to a surface comprising iron oxide.

Non-limiting examples of peptides in accordance with the above preferred embodiments of the first aspect are:

HRHRHRHRXR, HRHRXHRHRX, XHHRRHHRRH, HHRRXHHRRH, HRHRXHRHRX, XHHRRHHRRH, HHRRXHHRRH, HRHRHRHRYX, HRHRXHRHRY, XHHRRHHYRH, HHRRYHHRXH (SEQ ID NOs 1 to 11), wherein X is E or D and Y is A, C, F, G, I, L, M, N, P, Q, S, T, V, W or Y

The present invention relates in a second aspect to a (poly)peptide being tagged with the peptide of the first aspect.

A peptide has been defined herein above as a polycondensate of amino acids, the number of amino acids being 30 or less. A polypeptide (or protein) is accordingly a polycondensate of amino acids, the number of amino acids being 31 or more. The term “(poly)peptide” refers to both, peptides, and polypeptides.

The peptide of the first aspect is a peptide tag that can be fused to virtually any desired target (poly)peptide as fusion partner. By this fusion a fusion (poly)peptide is generated, and the peptide tag advantageously allows immobilizing the fusion (poly)peptide via the tag on metal oxide and silica surfaces.

Peptides in accordance with the invention may be fused N-terminally and/or C-terminally to a (poly)peptide. Also, it is envisaged to insert the sequence of a peptide in accordance with the invention into a (poly)peptide, such that on either side of the peptide of the invention there are flanking sequences, said flanking sequences originating from the fusion partner.

It is apparent from the description of the (poly)peptide of the second aspect that naturally occurring (poly)peptides do not fall under the term “(poly)peptide of the second aspect”.

Instead, there is the requirement that the peptide tag on the one hand and the above-mentioned fusion partner on the other hand are heterologous to each other.

It is furthermore envisaged to use linker sequences for inserting the peptide in accordance with the first aspect into said (poly)peptide of the second aspect. Preferably, such linker sequences, to the extent they are present, are heterologous with regard to said (poly)peptide used as fusion partner and furthermore heterologous with regard to the sequence of said peptide in accordance with the aspect. Preferred linker sequences include oligoglycine sequences and the sequence SSG. The linker may also be a cleavable linker. The use of cleavable linkers will allow the detachment of a desired protein (or no non-proteinaceous compound - see below) from, for example, an iron oxide containing surface, if desired.

The present invention also relates to conjugates, wherein a non-proteinaceous compound is tagged with the peptide of the first aspect. Conjugates are herein distinguished from fusion constructs. Accordingly, the moiety which is conjugated to a peptide in accordance with the present invention is a non-proteinaceous compound, such as an oligosaccharide, a polysaccharide, or a fatty acid. For instance, for sensing it the peptide tag of the invention might be coupled to a microorganism (e.g. bacteria) or virus prior to magnetic separation through tag binding, which might improve detection. In this case also proteinaceous material is coupled with the tag but also comprising non-proteinaceous compounds. This could particularly be interesting for the diagnosis of pathogenic microorganisms (e.g. bacteria) or viruses. Yet further, the tag can be used to couple magnetic nanoparticles to other polymers, which are e.g. stimuli-responsive for drug-delivery or which just can act as a protective layer around the nanoparticles. This can be helpful for applications, such as magnetic sensing, hyperthermia applications or imaging.

In accordance with a preferred embodiment of the second aspect the peptide of the first aspect is genetically fused or chemically linked to the (poly)peptide.

Since the fusion (poly)peptide entirely consists of amino acids also a corresponding fusion nucleic acid molecule can be produced which encodes the fusion (poly)peptide, preferably in expressible form. Such a fusion (poly)peptide is referred to herein as a genetically fused (poly)peptide.

The present invention therefore also provides a nucleic acid molecule encoding, preferably in expressible form at least one peptide of the first aspect and/or at least one (poly)peptide of the second aspect.

Such a nucleic acid molecule is preferably a vector. The term “vector” in accordance with the invention means preferably a plasmid, cosmid, virus, bacteriophage or another vector used e.g. conventionally in genetic engineering which carries a nucleic acid molecule encoding at least one peptide of the first aspect and/or at least one (poly)peptide of the second aspect. The nucleic acid molecule of the invention may, for example, be inserted into several commercially available vectors. Non-limiting examples include prokaryotic plasmid vectors, such as of the pUC-series, pBluescript (Stratagene), the pET-series of expression vectors (Novagen) or pCRTOPO (Invitrogen) and vectors compatible with an expression in mammalian cells like pREP (Invitrogen), pcDNA3 (Invitrogen), pCEP4 (Invitrogen), pMC1neo (Stratagene), pXT1 (Stratagene), pSG5 (Stratagene), EBO-pSV2neo, pBPV-1, pdBPVMMTneo, pRSVgpt, pRSVneo, pSV2-dhfr, pIZD35, pLXIN, pSIR (Clontech), pIRES-EGFP (Clontech), pEAK-10 (Edge Biosystems) pTriEx-Hygro (Novagen) and pCINeo (Promega). Examples for plasmid vectors suitable for Pichia pastoris comprise e.g. the plasmids pAO815, pPIC9K and pPIC3.5K (all Invitrogen).

The nucleic acid molecules inserted into the vector can e.g. be synthesized by standard methods, or partly isolated from natural sources. Ligation of the coding sequences to transcriptional regulatory elements and/or to other amino acid encoding sequences can also be carried out using established methods. Transcriptional regulatory elements (parts of an expression cassette) ensuring expression in prokaryotes or eukaryotic cells are well known to those skilled in the art. These elements comprise regulatory sequences ensuring the initiation of transcription (e. g., translation initiation codon, promoters, such as naturally associated or heterologous promoters and/or insulators; see above), internal ribosomal entry sites (IRES) (Owens, Proc. Natl. Acad. Sci. USA 98 (2001), 1471-1476) and optionally poly-A signals ensuring termination of transcription and stabilization of the transcript. Additional regulatory elements may include transcriptional as well as translational enhancers. Preferably, the polynucleotide encoding the polypeptide/protein or fusion protein of the invention is operatively linked to such expression control sequences allowing expression in prokaryotes or eukaryotic cells. The vector may further comprise nucleic acid sequences encoding secretion signals as further regulatory elements. Such sequences are well known to the person skilled in the art. Furthermore, depending on the expression system used, leader sequences capable of directing the expressed polypeptide to a cellular compartment may be added to the coding sequence of the polynucleotide of the invention. Such leader sequences are well known in the art.

Furthermore, it is preferred that the vector comprises a selectable marker. Examples of selectable markers include genes encoding resistance to neomycin, ampicillin, hygromycine, chloramphenicol, and kanamycin. Specifically designed vectors allow the shuttling of DNA between different hosts, such as bacteria-fungal cells or bacteria-animal cells (e. g. the Gateway system available at Invitrogen). An expression vector according to this invention is capable of directing the replication, and the expression, of the polynucleotide and encoded peptide or fusion protein of this invention. Apart from introduction via vectors such as phage vectors or viral vectors (e.g. adenoviral, retroviral), the nucleic acid molecules as described herein above may be designed for direct introduction or for introduction via liposomes into a cell. Additionally, baculoviral systems or systems based on vaccinia virus or Semliki Forest virus can be used as eukaryotic expression systems for the nucleic acid molecules of the invention.

The vectors preferably also contain a cleavage site for a restriction enzyme. Such cleavage site permits the insertion of a nucleic acid sequence encoding the peptide tag or (poly)peptide of the invention.

By contrast, a fusion (poly)peptide which is generated by a chemical link is not generated on the nucleic acid level but the peptide of the first aspect is actively linked to the (poly)peptide, for example by a peptide bond or by ethyl-dimethylaminopropyl-carbodiimide (EDC) coupling.

In accordance with a further preferred embodiment of the second aspect the (poly)peptide is an antibody or an antibody fragment, protein A, a hormone, an enzyme, a binding protein, a receptor, a ligand or a pharmaceutically active(poly)peptide.

The purification of these proteins and/or the use of these proteins in purification methods is of particular commercial interest. Such proteins are, for example, often recombinantly produced in cells or are isolated from their natural environment, e.g. from plants or animals. As discussed, the peptide tag of the invention facilitates the purification of the proteins from the discussed cells or natural environment.

The present invention relates in a third aspect to an inorganic surface comprising at least one peptide of the first aspect and/or at least one (poly)peptide of the second aspect directly bound to the inorganic surface.

With respect to the (poly)peptide of the second aspect it is to be understood that it is directly bound to the inorganic surface via the peptide of the first aspect which forms part of the (poly)peptide of the second aspect.

Said surface may have any geometry. It extends to planar and curved surfaces. A preferred geometry is particles, as will be further discussed herein below.

While it has been demonstrated in the examples that the peptide of the first aspect strongly binds to metal oxide (e.g. calcium oxide, magnesium oxide, titanium oxide, manganese oxide, nickel oxide, cobalt oxide) and silica surfaces it is believed that the peptide of the first aspect also strongly binds to further inorganic surfaces, such as surfaces of phosphates or carbonates.

The present invention also relates to uses of the surface in accordance with the third aspect, said uses being for a biotechnological process. In a preferred embodiment of said use, the use involves an enzyme and said biotechnological process is or comprises a reaction catalyzed by said enzyme.

Related thereto, the present invention also provides a method for enzymatic processing of a substrate, said method comprising: (a) bringing into a contact a surface in accordance with the third aspect, wherein said molecule is an enzyme, with a substrate of said enzyme. Preferably, said contacting is effected under conditions that permit conversion of said substrate into a product, the conversion being catalyzed by said enzyme. Such conditions are generally known in the art and may be specific for a given enzyme/substrate pair.

In a preferred embodiment of said method, said method further comprises one or more of the following: (b) separating said surface from the reaction mixture; (c) enriching or purifying the obtained product.

In another preferred embodiment, binding to said surface occurs in (a) 50 mM Tris buffer at pH 7.4, 137 mM NaCl and 2.7 mM KCI; and in (b) 50 mM PBS buffer at pH 7.4, 137 mM NaCl.

In accordance with a preferred embodiment of the third aspect the inorganic surface comprises iron oxide or silica.

As discussed herein above, it is demonstrated in the examples that the peptide of the first aspect strongly binds to iron oxide and silica surfaces. The binding to iron oxide is irreversible while the binding to silica is reversible under the conditions tested in accordance with the invention.

Among iron oxide and silica surfaces preference is given to iron oxide surfaces, especially magnetic iron oxide (nano)particles. Magnetic iron oxide nanoparticles are commercially available, e.g. from Sigma Aldrich. Silica surfaces are also available from Sigma Aldrich.

The term “iron oxide” includes any form of iron (Fe) oxide such as FeO, Fe₂O₃ and Fe₃O₄. Iron oxides may also be hydrated. Preferred are magnetic iron oxides, especially Fe₃O₄.

The term “silica” refers to any form containing silanol (Si—OH) or siloxane (Si—O—Si) groups. The silica is preferably underivatized silica, which is “naked” silica without any functional groups. Although silica has been frequently used as a stationary phase backbone matrix, direct adsorption of proteins on underivatized silica has not been widely exploited for industrial applications.³² Underivatized silica is very inexpensive (about 100 USD/L) when compared with other conventional chromatographic stationary phases (about 1000 USD/L). The average pore size of the silica to be used is preferably between 6 nm and of 50 nm.

The mentioned surface as well as the mentioned particles may consist of iron oxide or may comprise, in addition to iron oxide, other material such as oxides of other metals. Surfaces comprising mixtures of metal oxides and having magnetic properties are preferred. Preferred other metals are Co, Ni, Mn and Cu which other metals can also be comprised in small quantities only (dotation). Typical other materials are ferrites which have the following structure: XFe₂O₄, wherein X can be Co, Ni, Mn, Zn, Cu, Mg, Sr, Ba or mixtures of these elements.

In a preferred embodiment, magnetic nanoparticles are prepared in accordance with the methods described in the examples enclosed herewith.

In a preferred embodiment, increasing the length of the (W) region in a sequence having an (S)(W)(S) pattern, especially by increasing the number of Gs, while maintaining the overall number of amino acids in the two (S) regions constant, said amino acids in said (S) regions preferably being exclusively R or exclusively K, is a means of enhancing binding to surfaces containing iron oxide or silica.

In accordance with another preferred embodiment of the third aspect the inorganic surface is a particle, preferably a nanoparticle or a microparticle.

A “particle” is a small, localized object or entity comprising or consisting of inorganic material. The particle preferably has a spherical shape. The diameter of a nanoparticle or a microparticle is preferably in the range between 0.1 nm and 100 µm. The diameter of a nanoparticle is preferably between 1 and 50 nm, more preferably between 1 and 20 nm, even more preferably between 5 and 20 nm and most preferably between 5 and 15 nm. Preferred particles are colloidal particles.

Preferred silica particles are spherical particles including microparticles ranging from 10 nm to 100 µm.

In accordance with a more preferred embodiment of the third aspect (i) the iron oxide comprises ferrous oxide, magnetite, maghemite or a transition state between magnetite and maghemite, and/or (ii) the silica comprises silanol or siloxane.

The iron oxide magnetite is the chemical compound with formula Fe₃O₄. Controlled oxidation of Fe₃O₄ can be used to produce brown pigment quality γ-Fe₂O₃ (maghemite).

A silanol is a functional group in silicon chemistry with the connectivity Si—OH. A siloxane is a functional group with a Si—O—Si linkage.

The present invention relates in a fourth aspect to a microorganism or a host cell expressing the peptide of the first aspect and/or the (poly)peptide of the second aspect.

Such a microorganism or a host cell preferably comprises a nucleic acid molecule, e.g. a vector of the invention as described herein above.

The microorganism or a host cell may also be a genetically engineered microorganism or host cell expressing the peptide of the first aspect and/or the (poly)peptide of the second aspect from its genome. Means and methods for genetically engineering a microorganism or host cell are known in the art. The CRISPR-Cas technology is preferred since this technology allows to incorporate solely a nucleic acid sequence encoding the peptide of the first aspect and/or the (poly)peptide of the second aspect into the genome of a microorganism or a host cell.

The term “host cell” means any cell of any cell that is selected, modified, transformed, grown, or used or manipulated in any way, for the production of the peptide of the first aspect and/or the (poly)peptide of the second aspect by the cell.

The host cell or microorganism of the invention is typically produced by introducing the nucleic acid molecule of the invention into the host cell which upon its/their presence mediates the expression of the nucleic acid molecule of the invention encoding the peptide of the first aspect and/or the (poly)peptide of the second aspect. The host from which the host cell is derived or isolated may be any prokaryote or eukaryotic cell or organism, preferably with the exception of human embryonic stem cells that have been derived directly by destruction of a human embryo.

Useful microorganisms are, for example, those generally used for cloning and/or expression like E. coli(e.g., E coli strains BL21, HB101, DH5a, XL1 Blue, Y1090 and JM101), Salmonella typhimurium, Serratia marcescens, Burkholderia glumae, Pseudomonas putida, Pseudomonas fluorescens, Pseudomonas stutzeri, Streptomyces lividans, Lactococcus lactis, Mycobacterium smegmatis, Streptomyces coelicolor or Bacillus subtilis. Appropriate culture mediums and conditions for the above-described microorganisms are well known in the art. The microorganisms may also be yeast, such as Saccharomyces cerevisiae, Pichia pastoris or an Aspergillus. Preferred examples of microorganisms to be genetically engineered with the nucleic acid molecule of the invention are yeast, E. coli and/or a species of the genus Bacillus (e.g., B. subtilis).

The host cell is preferably a eukaryotic host cell. A suitable eukaryotic host cell may be a vertebrate cell, an insect cell, a nematode cell or a plant cell.

In a different preferred embodiment, the host cell is a mammalian host cell, such as a Chinese Hamster Ovary (CHO) cell, mouse myeloma lymphoblastoid, human embryonic kidney cell (HEK-293), human embryonic retinal cell (Crucell’s Per.C6), or human amniocyte cell (Glycotope and CEVEC). The cells are frequently used in the art to produce recombinant proteins. CHO cells are the most commonly used mammalian host cells for the industrial production of recombinant protein therapeutics for humans.

The present invention relates in a fifth aspect to a method for immobilizing the peptide of the first aspect and/or the (poly)peptide of the second aspect on an inorganic surface comprising contacting the inorganic surface with the peptide of the first aspect, the (poly)peptide of the second aspect and/or the microorganism or host cell of the fourth aspect under conditions, wherein the peptide of the first aspect and/or the (poly)peptide of the second aspect directly binds to the inorganic surface via the peptide of the first aspect.

The present invention relates in a sixth aspect to a method for purifying the peptide of the first aspect and/or the (poly)peptide of the second aspect comprising (a) contacting an inorganic surface with a mixture comprising the peptide of the first aspect, the (poly)peptide of the second aspect and/or the microorganism or host cell of the fourth aspect under conditions, wherein the peptide of the first aspect and/or the (poly)peptide of the second aspect directly bind(s) to the inorganic surface via the peptide according of the first aspect, and (b) optionally eluting the peptide of the first aspect and/or the (poly)peptide of the second aspect from the inorganic surface, thereby purifying the peptide of the first aspect and/or the (poly)peptide of the second aspect from said mixture.

In accordance with the fifth and sixth aspect, the microorganism or host cell expresses the peptide of the first aspect and/or the (poly)peptide of the second aspect during the method or has been cultured before carrying out the method, such that it already comprises the peptide of the first aspect and/or the (poly)peptide of the second aspect when the method of the fifth aspect is started. Both, the adherence of the inorganic surface to the inorganic surface as well as the elution thereof are considered purifications in accordance with the invention.

Given that the present invention provides peptides as well as molecules which are capable of binding to a surface-containing iron oxide, the skilled person can determine without further ado the conditions, wherein the peptide of the first aspect and/or the (poly)peptide of the second aspect directly bind(s) to the inorganic surface. The conditions are preferably in (a) 50 mM Tris buffer at pH 7.4, 137 mM NaCl and 2.7 mM KCl; and in (b) 50 mM PBS buffer at pH 7.4, 137 mM NaCl.

The inorganic surface is preferably an inorganic surface as has been described in connection with the previous aspects of the invention.

Once the peptide of the first aspect and/or the (poly)peptide of the second aspect directly binds to the inorganic surface the inorganic surface may be separated thereby also separating the (poly)peptide from a mixture. To the extent magnetic surfaces, magnetic particles and magnetic nanoparticles are used, said separating is preferably affected with a magnet. Devices for separating magnetic particles from mixtures are available from various manufacturers including Thermo Fisher.

The present invention relates in a seventh aspect to a kit comprising or consisting of (a)(i) a peptide in accordance with the first aspect; (ii) a (poly)peptide in accordance with the second aspect; and/or (iii) a nucleic acid encoding said peptide and/or (poly)peptide of (i) or a molecule of (ii), wherein said molecule is a polypeptide or protein, wherein said nucleic acid is preferably comprised in an expression vector.

In a preferred embodiment, the kit additionally comprises one or more or all of the following (a) a surface comprising iron oxide, preferably magnetic nanoparticles; (c) a solution or constituents for preparing said solution, said solution being capable of establishing conditions which allow immobilization of said peptide.

All amino acid sequences provided herein are presented starting with the most N-terminal residue and ending with the most C-terminal residue (N→C), as customarily done in the art, and the one-letter or three-letter code abbreviations as used to identify amino acids throughout the present invention correspond to those commonly used for amino acids.

Regarding the embodiments characterized in this specification, in particular in the claims, it is intended that each embodiment mentioned in a dependent claim is combined with each embodiment of each claim (independent or dependent) said dependent claim depends from. For example, in case of an independent claim 1 reciting 3 alternatives A, B and C, a dependent claim 2 reciting 3 alternatives D, E and F and a claim 3 depending from claims 1 and 2 and reciting 3 alternatives G, H and I, it is to be understood that the specification unambiguously discloses embodiments corresponding to combinations A, D, G; A, D, H; A, D, I; A, E, G; A, E, H; A, E, I; A, F, G; A, F, H; A, F, I; B, D, G; B, D, H; B, D, I; B, E, G; B, E, H; B, E, I; B, F, G; B, F, H; B, F, I; C, D, G; C, D, H; C, D, I; C, E, G; C, E, H; C, E, I; C, F, G; C, F, H; C, F, I, unless specifically mentioned otherwise.

Similarly, and also in those cases where independent and/or dependent claims do not recite alternatives, it is understood that if dependent claims refer back to a plurality of preceding claims, any combination of subject-matter covered thereby is considered to be explicitly disclosed. For example, in case of an independent claim 1, a dependent claim 2 referring back to claim 1, and a dependent claim 3 referring back to both claims 2 and 1, it follows that the combination of the subject-matter of claims 3 and 1 is clearly and unambiguously disclosed as is the combination of the subject-matter of claims 3, 2 and 1. In case a further dependent claim 4 is present which refers to any one of claims 1 to 3, it follows that the combination of the subject-matter of claims 4 and 1, of claims 4, 2 and 1, of claims 4, 3 and 1, as well as of claims 4, 3, 2 and 1 is clearly and unambiguously disclosed.

The above considerations apply mutatis mutandis to all appended claims.

The figures show.

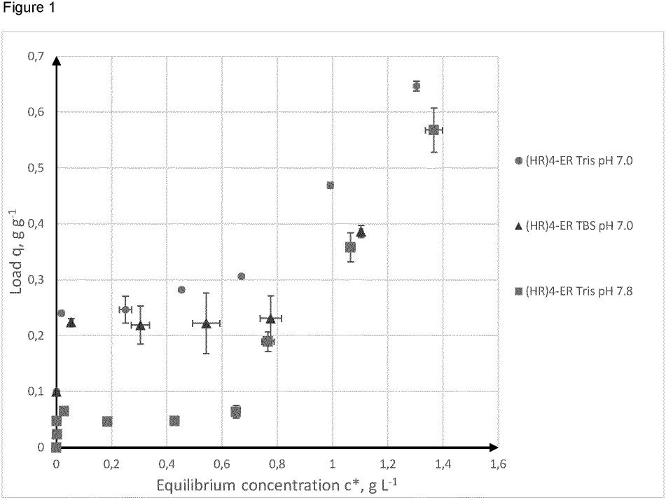

FIG. 1. Adsorption isotherm of (HR)₄-tagged ER with 1 g L⁻¹ magnetic nanoparticles in three different environments: 50 mM Tris pH 7.0,50 mM Tris pH 7.8 and TBS pH 7.0. Analysis via BCA. Measured as technical duplicates.

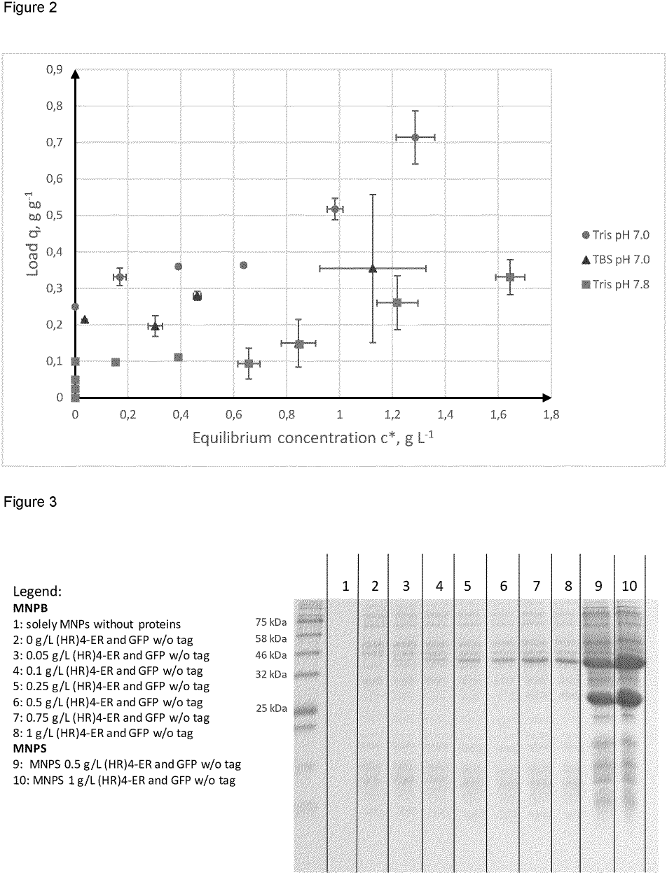

FIG. 2. Adsorption isotherm of (HR)₄-tagged ER with 1 g L⁻¹ magnetic nanoparticles in three different environments: 50 mM Tris pH 7.0,50 mM Tris pH 7.8 and TBS pH 7.0.. Analysis via HPLC. Measured as technical duplicates.

FIG. 3. Competitive adsorption isotherm of (HR)₄-ER with GFP w/o tag and a constant E. coli lysate concentration of 2 g L⁻¹ and 1 g L⁻¹ IONs. 10 µL of a final ION concentration of 2 g L⁻¹ was loaded onto the gel (20 µg). The ER amount on the IONs increased whereas no GFP was adsorbed. Lysate proteins are adsorbed being constant over the whole range. References (Supernatant of ION incubation (IONS)) in lane 9 and 10 show both proteins existent in the mixture.

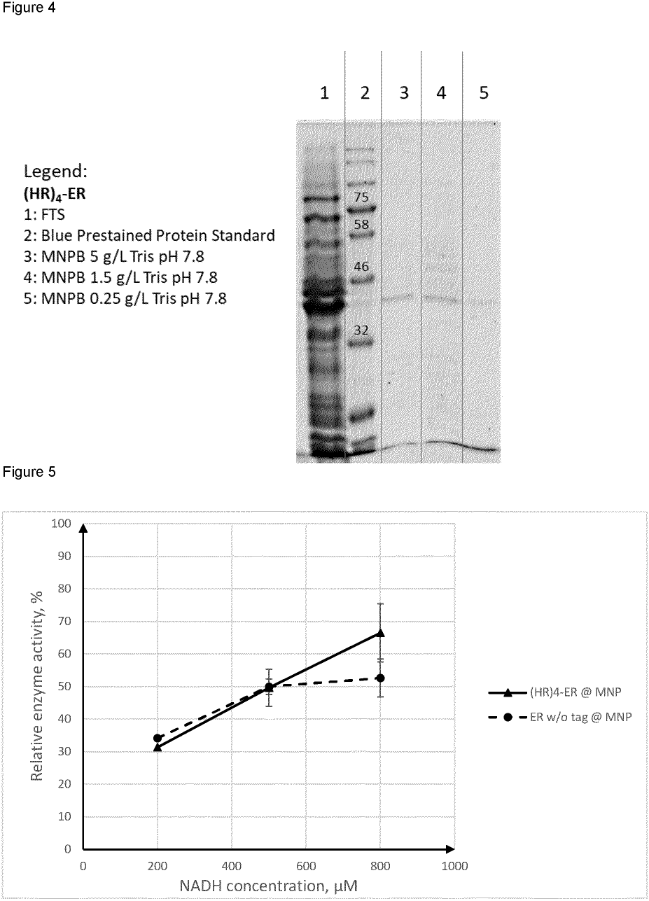

FIG. 4. Binding of (HR)₄-ER to IONs. Freeze-thaw supernatant (FTS) after 3 times freeze-thaw. Incubation of FTS with different magnetic iron oxide nanoparticle (ION) concentrations. IONs were resuspended in Tris pH 7.8. MNPB: bound proteins to ION. (HR)₄-ER detected in MNPB fractions. MNPB normalized to 5 g L⁻¹. The numbers in lane 2 are the sizes of the underlying band in kDa.

FIG. 5. Relative enzyme activity of immobilized (HR)₄-ER and ER w/o tag onto IONs. The relative enzyme activity is calculated as the adsorbed enzyme activity divided by the free enzyme activity. The activity was determined at 10 mM maleimide and different concentrations of NADH at 50 mM Tris pH 7.0. The adsorption happened at 50 mM Tris pH 7.0. Measurements performed as technical triplicates.

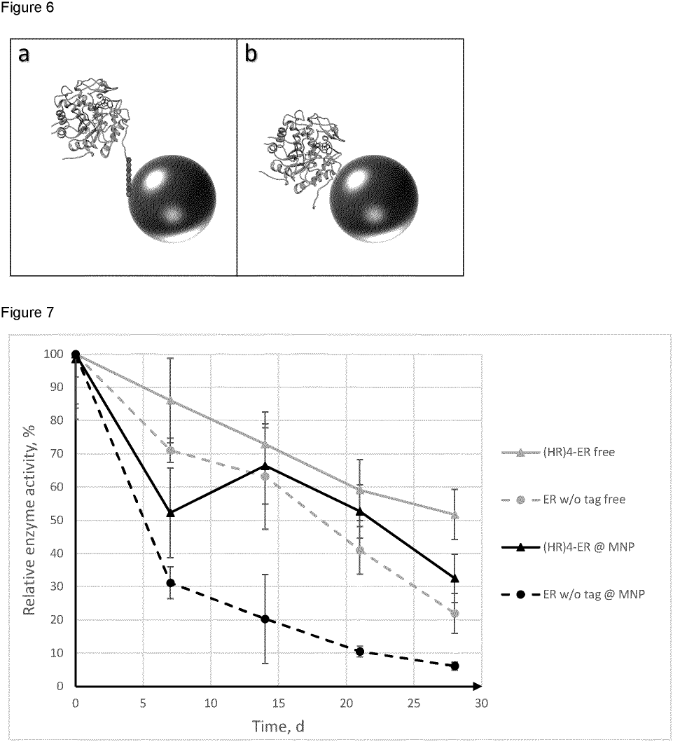

FIG. 6. Model for adsorption of (HR)₄-ER and ER w/o tag to IONs. (HR)₄-ER adsorbs to IONs via tag whereas ER w/o tag adsorbs unspecifically.

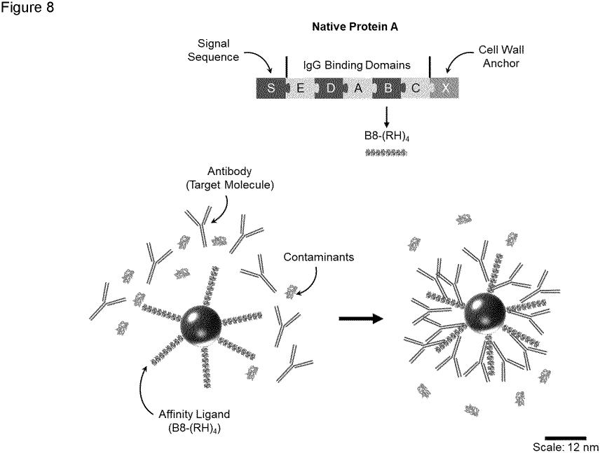

FIG. 7. Long-term stability of ER w/o tag and (HR)₄-ER in a free and immobilized state, respectively. The enzyme activity was determined at 10 mM maleimide, 0.5 mM NADH and 50 mM Tris pH 7.0 for 28 days. The enzyme activity was related to the enzyme activity of the corresponding sample at the timepoint 0. The enzyme activities were measured every 7 days as technical triplicates. The samples were stored at 4° C.

FIG. 8. Principle of IgG-binding by novel Protein A-based ligand modified, bare IONs.

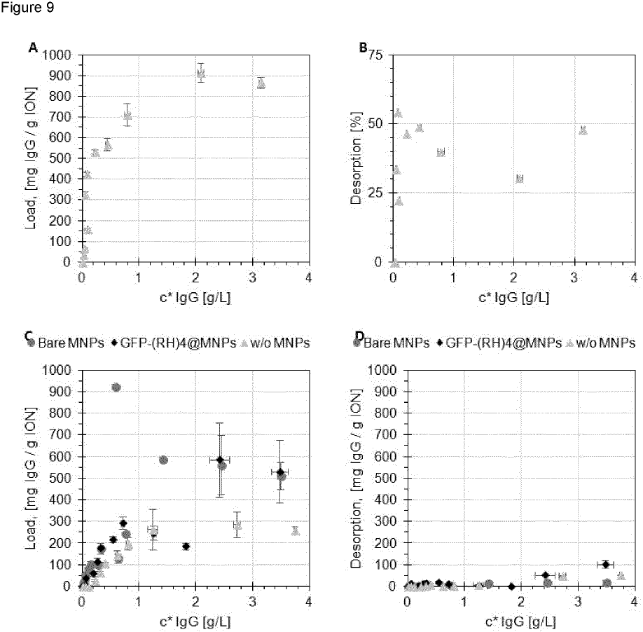

FIG. 9. IgG binding studies using IONs@B8-(RH)₄ (A / B) and diverse control systems (C / D). Bare IONs, GFP-(RH)₄@IONs, without (w/o) IONs are used as references. Binding conditions (A / C): 20 mM phosphate pH 7.4, 150 mM NaCl, 1 h, 25° C. Desorption conditions (B / D): 50 mM glycine pH 2.9, 150 mM NaCl, 2 h, 25° C. All measurements were conducted as technical duplicates.

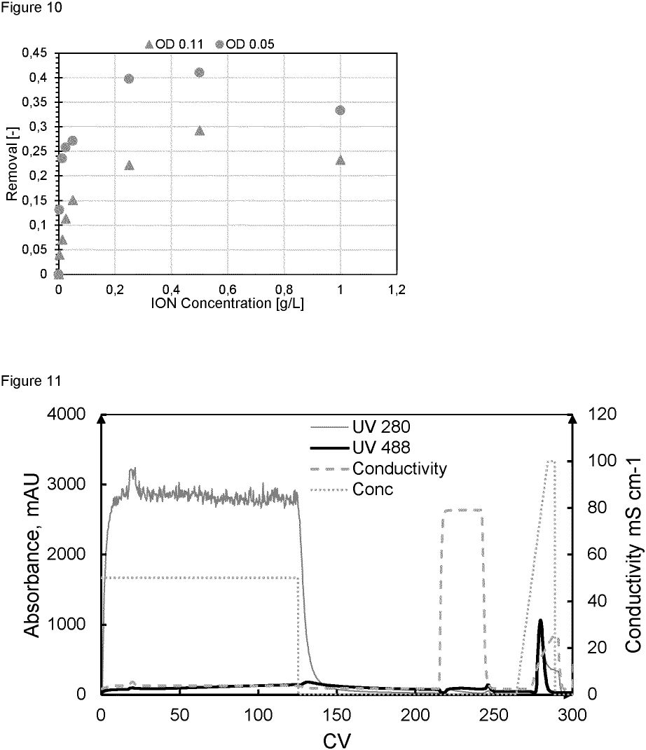

FIG. 10. Cell capture of E. coli by functionalized IONs (ION@B8-RH₄@IgG) and using two different initial E. coli concentrations.

FIG. 11. Purification of GFP-(RH)₄ with a silica stationary phase and 50 mM TRIS pH 8 mobile phase. The purification is performed at a flow rate of 76 cm h⁻¹. In the first 125 CV the lysate is loaded on the column. After loading the column is equilibrated till the 280 nm curve shows no protein flow through. A washing step with 1 M NaCl in 50 mM TRIS pH 8 is performed for 25 CV. The elution is performed with a 0.5 M lysine in 50 mM TRIS pH 8 buffer via a gradient over 20 CV.

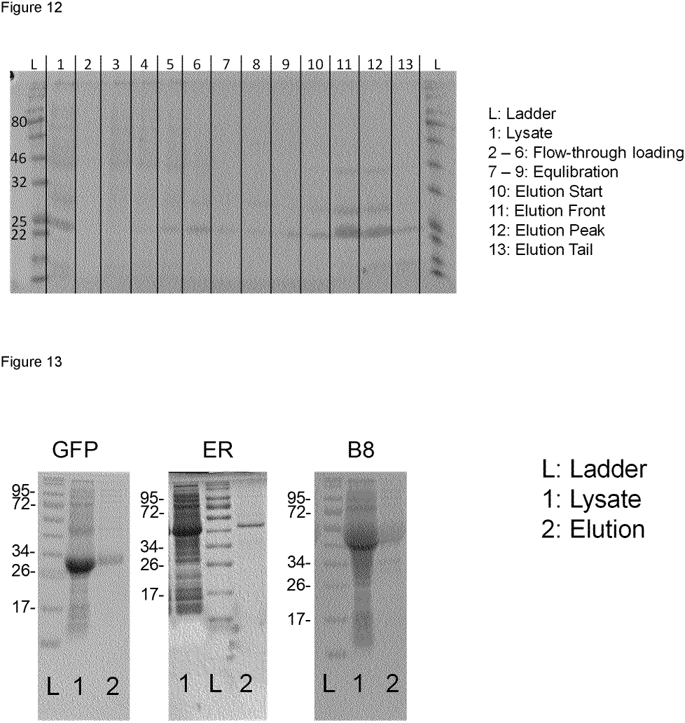

FIG. 12. SDS-PAGE (12.5%) of the GFP-(RH)₄ Purification with silica stationary phase. GFP-(RH)₄ can be seen slightly over the 25 kDa ladder band in the lysate and the elution lanes. The flow-through lanes show increasing GFP-(RH)₄ concentrations which indicates overloading of the stationary phase. Gel evaluation with the Amersham Typhoon resulted in a purity of about 90 % for the Elution Peak (Lane 12).

FIG. 13: For each protein (green fluorescent protein (GFP), ene reductase (ER) and polymerized B binding domains of protein A (B8)) production process, a SDS-PAGE is shown containing a ladder with marker proteins indicating distinct sizes (left), the lysate (middle) and the purified protein (right) is shown (For ene reductase the ladder and the lysate are switched). All proteins contain an (RH)₄-tag and were loaded on a silica column with a loading buffer (TRIS 50 mM pH 8) and eluted with a similar buffer containing additional 500 mM lysine. Densitometric determination yields purities between 80-85% after just one purification step.

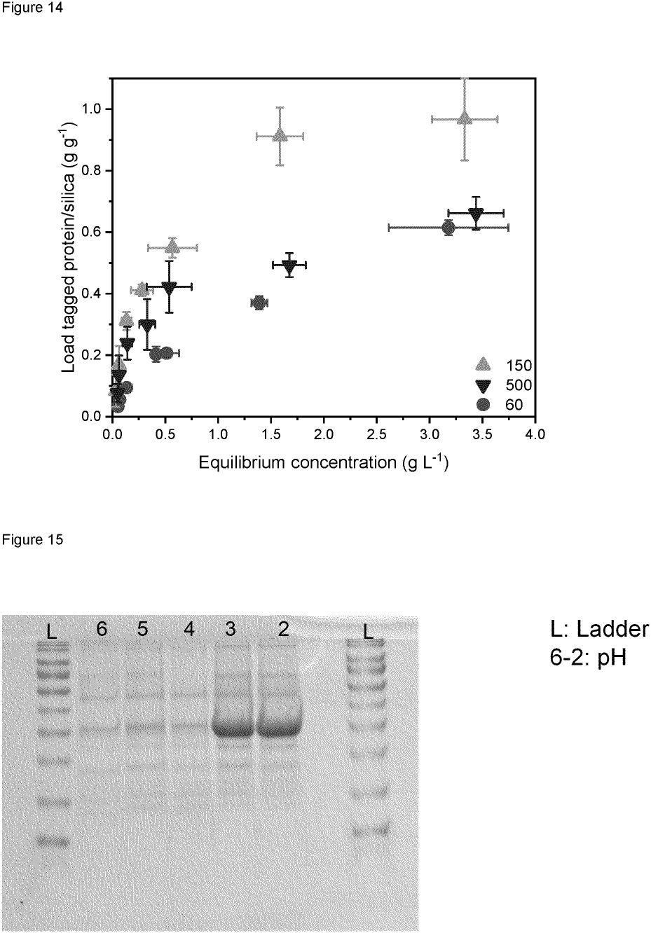

FIG. 14: Adsorption isotherms of tagged ene reductase to different silica species (the number in the legend indicates the pore size in Å).

FIG. 15: The “on particle” SDS-PAGE shows the proteins bound to the silica resin material after the pH shift to pH 6, 5, 4, 3 or 2 and elution with lysine (500 mM). The first and the last line represent protein ladders.

The examples illustrate the invention.

### EXAMPLE 1 - MATERIAL AND METHODS

### Synthesis of IONs

The iron oxide nanoparticles used for this study were synthesized by co-precipitation of Fe²⁺ and Fe³⁺ in alkaline aqueous solutions adapted to the procedure described by Roth et al. (Roth et al. 2015). Briefly, 21.2 g of FeCl₃ x 6 H₂O and 8.29 g of FeCl₂ x 4 H₂O were dissolved in 200 mL of deionized, degassed water which equals a Fe(III) : Fe(II) ratio of 1.88 : 1. This iron chloride solution was added to 1 L of a 1 M solution of NaOH prepared with deionized, degassed water stirring at 250 rpm in a reaction vessel. The reaction mixture was kept under nitrogen atmosphere at 25° C. and stirred for 30 more minutes before the nanoparticles that had formed were washed several times with degassed deionized water until the conductivity of the ION solution was below 200 µS cm⁻¹. In order to separate the particles from the washing water the mixture was placed on a NdFeB permanent magnet. FeCl₃ x 6 H₂O and sodium hydroxide were purchased from AppliChem GmbH, Germany in the highest purity available. FeCl₂ x 4 H₂O extra pure was obtained from Merck KGaA, Germany.

Silica beads have been purchased from Sigma-Aldrich (Davisil 643).

### Protein Immobilization

### Adsorption Isotherm

Adsorption isotherms were conducted to investigate the affinity and maximum load of pure (HR)₄-ER to IONs. The (HR)₄-ER was considered as pure if the purity was above 95% according to densitometric analysis via SDS-PAGE. Different (HR)₄-ER concentrations were mixed with IONs obtaining 1 g L⁻¹ ION in every sample with different final protein concentrations. These different protein concentrations were 0, 0.025, 0.05, 0.1, 0.25, 0.5, 0.75, 1.0, 1.5 and 2 g L⁻¹. The mixtures were incubated for 1 hour at 16° C. and 1200 rpm. The IONs were separated from the supernatant by applying a magnetic field. This supernatant was defined as MNPS (ION supernatant). The IONs were washed with the same buffer. Therefore, the same volume as before was added to the IONs and mixed by vortexing. One more time, a magnetic field was applied separating the IONs from the supernatant. That supernatant was termed as MNPW (ION wash). Finally, the IONs were resuspended in the initial volume, defining this sample as MNPB (ION bound).

The MNPS and MNPW samples were analyzed by BCA assay and by HPLC (see HPLC analysis). Additionally, the enzymatic activity of MNPS, MNPW and MNPB samples was investigated as described below. All samples were conducted as technical duplicates. Finally, the values were fitted using the Langmuir fit.

### HPLC Analysis

Besides the BCA analysis, the MNPS and MNPW samples of the adsorption isotherm were analyzed via HPLC. These samples were centrifuged (17000 g, 5 min) prior analysis. 8 µL of each sample were injected and analyzed on a C4 column (Aeris 3.6 µm Widepore C4 200 Å 150 × 2.1 mm). A mixture of water with 20 mM TFA (Buffer A) and 100% acetronitrile with 20 mM TFA (Buffer B) was used as mobile phase. Following method was applied: Firstly, a gradient going from 40% B to 60% B and a gradient length of 10 CV was applied, followed by a 100% B step which lasted 3 CV. Finally, the column was equilibrated at 40% B for 5 CV. The samples were injected two times. The amount of (HR)₄-ER was calculated by a standard curve of (HR)₄-ER standards in the corresponding buffers.

### Capturing (HR)₄-ER with IONs (Qualitative Analysis)

The binding of (HR)₄-ER from the lysate was detected by SDS-PAGE. For that, the E. coli cells were lysed after cultivation in shaking flasks via freeze-thaw. The IONs and the pellet of an (HR)₄-ER cultivation were resuspended in 50 mM Tris pH 7.8. The supernatant after freeze-thaw (FTS) was mixed 1:1 with different ION concentrations to obtain final ION concentrations of 5, 1.5 and 0.25 g L⁻¹, respectively. The mixtures were incubated for 1 hour at 10° C. and 1000 rpm. A magnetic field was applied to separate the IONs from the supernatant. This supernatant was defined as MNPS. Washing the IONs with the same buffer, the same volume as before was added to the IONs, mixed by shortly vortexing and again a magnetic field was applied separating the IONs from the supernatant. That supernatant was termed as MNPW. Finally, the IONs were resuspended in defined volumes to obtain normalized ION concentrations (MNPB, ION bound) and to compare the binding by SDS-PAGE.

### Competitive Adsorption Isotherm

The goal of the competitive adsorption isotherm was to show the adsorption of a (HR)₄-tagged protein in a highly competitive environment containing many other proteins and one protein with the same concentration. For this, every sample contained a constant concentration of 2 g L⁻¹ E. coli lysate proteins and 1 g L⁻¹ IONs. The concentrations of (HR)₄-ER and GFP w/o tag were added to this mixture and their concentrations were varied using 0, 0.05, 0.1, 0.25, 0.5, 0.75 and 1 g L⁻¹. All components were prepared in 50 mM Tris pH 7.0. GFP w/o tag and (HR)₄-ER had a purity above 95% according to densitometry. All experiments have been performed in technical triplicates. The samples were incubated at 16° C. for 1 h at 1200 rpm. The following steps were performed analogously to the adsorption isotherm of the pure (HR)₄-ER. To show which proteins adsorbed to the IONs, the MNPB samples were concentrated to 4 g L⁻¹ IONs each and mixed 1:1 with SDS loading buffer containing DTT. That mixture was boiled for 5 min at 95° C. and 10 µL were loaded onto a 15% polyacrylamide gel.

### Measurement of Enzymatic Activity

The enzymatic activity of ene reductase was assessed by measuring the decrease of absorption at 340 nm which correlates with the decrease of NADH which is oxidized to NAD⁺. Hereby, the reaction rate of the enzyme at 500 µM NADH (or 200 and 800 µM) and 10 mM maleimide in 50 mM Tris pH 7.0 at 30° C. was determined. The free enzyme had a final concentration of approximately 1 - 5 µg mL⁻¹ and the immobilized variant 10 - 20 µg mL⁻¹ to assure a linear decrease of the absorption. The extinction at 340 nm was determined every 40 s for 10 min. As blank served the mixtures without maleimide. Technical triplicates of each sample were measured in triplicates.

The enzyme activity was calculated as Units (turnover of 1 µmol substrate per min). So, the specific enzymatic activity vₓ (in U mg⁻¹) was calculated from the decreasing absorption ΔA₃₄₀ by applying the Lambert-Beer law and considering the reaction volume V_{R} (in L) and applied enzyme mass m_{E} (in g).

$${v}_{x} = \frac{Δ {A}_{340} \text{} ⋅ {V}_{R}}{Δ t \text{} ⋅ {ε}_{N A D H} \text{} ⋅ d ⋅ {m}_{E}}$$

The molar extinction coefficient of NADH ε_{NADH} is 6220 L mol⁻¹ cm⁻¹ and the thickness d is 0.59 cm. Only a linear decrease of absorption was used for the calculation. For this case, linear means the linear decrease of absorption for at least 3 min (6 time points) showing an R²> 0.995. The “activity” of the blank was fitted over the whole 10 min since no linear fit with R² > 0.995 was possible. The blank was subtracted from the enzyme activity of the sample.

The relative enzyme activities of the immobilized variants were calculated as the specific enzyme activity of the immobilized variant divided by the specific enzyme activity of the free variant. Regarding the long-term stability, the enzyme activity of the free ER and immobilized ER variants were measured every 7 days for 28 days. The variants were stored at 4° C. The experiments were performed as technical triplicates and each replicate measured three times. The relative enzyme activity was calculated from the specific enzyme activity at the time point 0 divided by the specific enzyme activity at the time point x.

### EXAMPLE 2 - RESULTS

Ene reductase is an enzyme from the class of oxidoreductases which catalyzes the reduction of activated alkenes. This enzyme was tagged with a (HR)₄-tag. Since GFP tagged with this (HR)₄-tag adsorbed to magnetic iron oxide nanoparticles quite well, it was aimed to demonstrate its transferability to other more complex proteins. That would demonstrate the broad applicability of the tag. The tag consists of histidine and arginine showing strong interactions with iron oxide nanoparticles. Previous adsorption studies of GFP bearing different tags have been performed at Tris and a physiological pH. Thus, adsorption studies of (HR)₄-tagged ene reductase were made at similar conditions. The following figures show the adsorption isotherms of (HR)₄-ER with 1 g L⁻¹ magnetic nanoparticles. These adsorption isotherms were analyzed via BCA and HPLC, respectively.

The adsorption isotherms analyzed via BCA and HPLC showed the same trends. For better comparison with loads from the literature, the curves were fitted with Langmuir considering the range from 0 to 0.75 g L⁻¹ (Tris pH 7.8) or 1 g L⁻¹ equilibrium concentration (Tris and TBS pH 7.0). The highest adsorption could be measured at 50 mM Tris pH 7.0 having a maximum load of 0.28 ± 0.008 g g⁻¹ (BCA) or 0.383 ± 0.009 g g⁻¹ (HPLC), respectively. At TBS pH 7.0 the load was 0.23 ± 0.042 g g⁻¹ (BCA) or 0.243 ± 0.009 g g⁻¹ (HPLC). The lowest load was assessed for 50 mM Tris pH 7.8 with 0.058 ± 0.006 g g⁻¹ (BCA) and 0.114 ± 0.025 g g⁻¹ (HPLC).

FIG. 3 depicts that with increasing GFP w/o tag and (HR)₄-ER concentration only a band at around 40 kDa is getting stronger and no band at 28 kDa. This means that (HR)₄-ER (41.02 kDa) is binding and no GFP w/o tag (28.20 kDa), although they are present in the same concentrations and they are both negatively charged (pl_{GFP} = 5.37; pl_{(HR)4-ER} = 6.2). Lanes 9 and 10 served as reference to show the existence of both proteins in the mixture.

These findings could be supported by another experiment. In that experiment, (HR)₄-ER was captured directly from an E. coli cell lysate via IONs (see FIG. 4). For this, lysate was incubated with IONs at different concentrations and a binding of (HR)₄-ER could be observed qualitatively whereas less lysate proteins adsorbed on the IONs.

Therefore, it is assumed that the tag is responsible for a strong interaction and outcompetes other proteins not having the tag. The experiment of the competitive adsorption isotherm and the capturing experiment together confirmed the binding because of the tag.

The strong binding and high affinity of the (HR)₄-tag suggest its use as immobilization tag for enzymes. One important prerequisite for enzyme immobilization is the retained activity of an enzyme despite its immobilization. The relative enzyme activity of ION-immobilized (HR)₄-ER and ER w/o tag were determined after mixing pure ER with IONs.

FIG. 5 depicts the relative enzyme activity as a function of NADH concentration. The relative enzyme activity increases for ER w/o tag and (HR)₄-ER with increasing NADH concentration. This phenomenon is presumably caused by the higher probability of a collision of NADH and enzyme when more NADH is present which in turn leads to a reaction. More importantly, ER is still active after immobilization and supports the use of IONs as carrier for immobilization purposes. A few research groups have also published an immobilization of enzymes to IONs.^{18,21-23} Both enzymes exhibit the same relative enzyme activity, thus no advantage is seen for the tag under those conditions. Nevertheless, it seems that the immobilized ER w/o tag saturates at around 0.8 mM NADH whereas the adsorbed (HR)₄-ER does not. This would indicate a more “directed” adsorption of (HR)₄-ER to the IONs while ER w/o tag just binds to the IONs electrostatically and in a more undirected way what would limit the mass transport to the active center of the enzyme. The hypothesis is that due to adsorption via (HR)₄ tag the active center is believed to be more accessible for substrates than the active center of the ER w/o tag which binds non-specifically and maybe oriented its catalytic center towards the IONs. This is depicted in FIG. 6.

The activity of both ER variants and both states (free, immobilized) decreased. Interestingly, the relative enzyme activity of immobilized ER decreased faster than their free counterparts. More importantly, the ER w/o tag bound to IONs showed the fastest decrease in relative enzyme activity. The tag seems to have a stabilizing effect onto the free enzyme and the immobilized one. The immobilized (HR)₄-tagged ER retained 32.5 % of its initial activity whereas the immobilized ER w/o tag showed only 7.9 %. These results indicate that the ER w/o tag is less stable on the IONs. One possibility is that the untagged variant binds to the IONs nonspecifically and is distorted over time while the (HR)₄-ER preferentially adsorbs via tag and is less affected by particles.

Another interesting field for protein immobilization is the immobilization of protein domains for antibody purification. A prominent example for the use of this strategy is Protein A chromatography.

### EXAMPLE 3 - ANTIBODIES

Antibodies are important molecules, that are used in diagnostics, sensor technology and as therapeutics inter alia in the field of hematology and oncology.^{24,25} For their purification, a general platform process is applied. Thereby, Protein A-chromatography is the central step of this process. This step ensures capturing of the antibody, reducing the whole process volume, and resulting in purities over 95% while yields of 99% are achieved. However, during the whole antibody manufacturing, the downstream process accounts for the biggest part of the costs with the Protein A-chromatography owning the biggest share.²⁶⁻²⁸

This is especially due to the rate limiting step of pore diffusion. This is a general problem of chromatography, but Protein A being a big-sized ligand (compared to e.g. ion exchange ligand) and the target molecule IgG being likewise big-sized exacerbate the problem with IgG’s low effective diffusivity. This leads to the need of high residence times and thus lower productivities.²⁹ Magnetic separation with non-porous magnetic iron oxide nanoparticles (IONs) has the potential to overcome these problems. Due to their nanoscale size, they offer high specific surface areas (~89 m²/g) without the need of pores.³⁰ For protein immobilization, usually functionalized magnetic nanoparticles e.g. by functional silanes or metal chelate materials are used. This approach differs from others: unfunctionalized IONs are used for a Protein A derivative consisting of 8 polymerized B-domains.³¹ The ligand’s terminus is functionalized with the affinity peptide tag (RH)₄. FIG. 8 shows this ligand and the principle. These affinity materials could be used to efficiently bind and elute human IgG.

### Methods

The polymerized B-domain protein (B8-(RH)₄) was placed in the plasmid pET28a and expressed in E. coli BLR(DE3). The intracellularly produced protein was released by the method described in (Kaveh-Baghbaderani et al., 2018). The supernatant obtained in this way was purified by immobilized metal ion affinity chromatography (IMAC, HiTrap FF; GE Healthcare) to a purity >95% (confirmed by SDS-PAGE). For functionalization, 40 mg/mL B8-(RH)₄ was incubated with 0.5 g/L IONs in 20 mM Tris pH 7.0 with 150 mM NaCl at 25° C. for 1 h while shaking. Performing the BCA-assay (Thermo Fisher Scientific GmbH) onto these particles revealed a ligand density of 19.8 mg/g IONs. Human polyclonal IgG (Gammanorm, Octapharma GmbH), that was previously purified by Protein A-chromatography (Bio-Scale SuprA, Bio-Rad GmbH) in order to remove non-binding subclasses of IgG, was used for equilibrium isotherms. The ION@B8-(RH)₄ were incubated with different concentrations (up to 4 g/L) human polyclonal IgG (Gammanorm, Octapharma GmbH) for 1 h at 25° C. in 20 mM PBS (pH 7.4, 150 mM NaCl) while shaking. IgG was eluted by 50 mM Glycine pH 2.9 with 150 mM NaCl for 2 h at the same incubation conditions. IgG was quantified by BCA-assay. E. coli cells were separated by IONs@B8-(RH)₄ that were further functionalized with bound anti E. coli antibodies (Bio-Rad, Germany) in a concentration of 25 mg per g IONs. The bacteria cells (BL21(DE3) have been labeled by GFP during expression induced by 25 µM IPTG. Therefore, the gene was inserted into the pET28a vector. Varying concentrations of MNP@B8-RH4@rabbitlgG were shaken with E. coli set to an optical density 0.05 and 0.1 at 550 rpm for 30 min. The particles were resuspended and magnetically separated. The fluorescence of the supernatant was measured in a microscale thermophoresis device (Monolith NT.115, NanoTemper, Germany, fluorescence only). The E. coli removal is the ratio of the initial fluorescence to the fluorescence after incubation.

### Results

FIG. 9, A and B shows adsorption isotherms of IgG and their elution dependent on the equilibrium concentration c*. This novel material results in unprecedented IgG binding capacities of up to 915 mg IgG per g IONs. Thereby, up to 55% of the bound IgG could be successfully desorbed. In order to prove the specificity of these particles, various control systems were examined for their binding and elution capabilities for antibodies (FIGS. 9, C and D): (i) non-functionalized IONs to detect unspecific binding to the iron oxide surface, (ii) IONs functionalized with a model protein (GFP), also via the affinity tag (RH)₄, (iii) completely without IONs to see wall adsorption effects. The binding of the latter control was related to the same (non-existent) amount of IONs for better comparability. It becomes clear that any kind of surface - be it a nanoscale particle system or vessel walls - is capable of binding antibodies. However, the pH-shift to below 3 is not sufficient to elute them again. It was thus possible to prove that the functionality of the materials presented above is specifically based on the binding to Protein A derivatives.

These ligand functionalized IONs must not only be used for purification but can also be used for cell separation and cell detection. As an example, the B8-(RH)₄@IONs can be bound to anti-E. coli rabbit IgG. These IONs have been used for E. coli (BL21(DE3)) separation. Since the cell serves as a model, it was intracellularly labeled by GFP during protein expression so the cell removal could be monitored by fluorescence depletion (device: Monolith NT.115, NanoTemper Technologies GmbH, Germany). FIG. 10 shows the successful cell separation by these IONs using two different starting optical densities (OD) of E. coli. Up to 41% of an E. coli suspension (OD = 0.05) could be removed. These IONs could be used for cell separation, concentration, sorting and detection. The latter could be achieved e.g. by enzyme-linked reactions or using fluorescent labelings.

### EXAMPLE 4 - PURIFICATION OF TAGGED PROTEINS WITH SILICA-BASED COLUMN CHROMATOGRAPHY

### Materials and Methods

TABLE 1

Used Bacterial strains

| | |
|---|---|
| Strain | Genotype |
| E. coli BL21 DE3 | F- ompT gal dcm Ion hsdSB(rB- mB-) λ(DE3[lacl lacUV5-T7 gene 1 ind1 sam7 in5]) |

TABLE 2

Buffers and solutions

| | |
|---|---|
| Name | Composition |
| Washing buffer 1 (WB1) | 50 mM Tris |
| | pH 8 |
| Washing buffer 2 (WB2) | 50 mM Tris |
| | 1 M NaCl |
| | pH 8 |
| Elution buffer 1 (E1) | 0.5 M Lysine |
| | 50 mM Tris |
| | pH 8 |
| Lysogeny broth medium (LB) | 1% Tryptone |
| | 0.5% Yeast Extract |
| | 0.5 % NaCl |
| Fairbanks A (Staining solution for SDS gels) | 25% Isopropanol |
| | 10% Acetic Acid |
| | 0.05% Coomassie R-250 |
| Fairbanks D (Destaining solution) | 10% Acetic Acid |

The E. coli were already containing the plasmid with the (RH)₄-GFP Tag. A pre-culture was prepared with the cells using LB medium. A concentration of 100 µg/mL of Ampicillin was added to the culture, to ensure that only the cells with the correct plasmid were growing in the medium. After the addition of the appropriate antibiotic to the medium the pre-culture was transferred to the final 1 L baffled flasks, containing 350 mL medium. The final culture volume was approximately 400 mL. These were incubated at 37° C. at 200 rpm until the OD₆₀₀ reached 0.6 - 0.7. This step was followed by the induction with 1 µM IPTG and the overnight incubation at 37° C.

After overnight expression, the cells were harvested via centrifugation (3200 rpm, 15 min, 8° C.). The cell pellet was re-suspended in WB 1. To ensure the stability of the (RH)₄-GFP and removal of remaining DNA, protease inhibitor and DNAse I was added to the re-suspended solution. The suspension was put on ice and lysed via sonication (max. amplitude 40%, 3 min - 10 s on 20 s off). The lysed cell suspension was centrifuged to remove cell particles from the lysate. The centrifugation was carried out at 7927 rpm for 35 min at 4° C.

The cleared lysate was sterile filtered using a bottle top filter (pore diameter 0.2 µm) to remove remaining cell debris, DNA and other contaminants.

### Chromatography

Chromatography experiment was conducted using an Aekta Purifier by GE Healthcare Life Sciences. An Omnifit Labware column (d= 0.7854 cm) was used and filled with wide pore silica slurry (Davisil 643, Sigma), resulting in an overall column volume of 1 mL. The HETP and asymmetry factor was measured using 1 M NaCl solution and measuring the peak of the conductometry. The column was loaded with 50% lysate and 50% 50 mM TRIS pH 8 at a flow rate of 1 mL min⁻¹. Following the loading of the column, it was washed with WB1 for 5 CV until the curves of 260 nm and 280 nm reached approximately 0 mAU to ensure that no further protein was passing the column. The column was cleared of unspecifically bound proteins with WB2 for 5 CV. The higher salt concentration removes ionically bound proteins. The column was then washed for 5 CV with WB1 before the start of the elution with E1 by a gradient from 0 to 100%.

### SDS-Page

A SDS-PAGE separates charged molecules in a mix according to their electrophoretic properties in a polyacrylamide gel by their differences in molecular weight in an electric field. SDS as an anionic detergent can denature proteins up to their tertiary structure and apply a negative charge in a relative proportion to the proteins’ mass. The negatively charged and linearized polypeptides will move through the gel towards the anode. Proteins that are bigger in size will move slower than smaller ones.

For the analysis of (RH)₄-GFP a 12.5% SDS gel was prepared as follows:

TABLE 3

Conditions for SDS-gel

| | |
|---|---|
| Volume | Solution |
| 3.3 mL | ddH₂O |
| 2.5 mL | 40% Acrylamide |
| 2 mL | 1.5 M Tris pH 8.8 |
| 80 µL | 10% SDS |
| 80 µL | 10% APS |
| 8 µL | TEMED |

The samples were incubated with 5x Laemmli buffer at 95° C. for 5 min. In addition to the samples, a protein standard (color pre-stained protein standard, NEB 11-245 kDa) was added. After the electrophoresis, the gels were stained Fairbanks A and de-stained with Fairbanks D. The bands became visible because the stain would embed itself into the alkaline sidechains of the amino acids. The evaluation of the gel was performed after scanning the gel with an Amersham Typhoon scanner (GE Healthcare, Great Britain).

The purification of GFP-HR4 with silica stationary phase is shown to be possible. During the loading, the 280 nm curve clearly shows the flow through of proteins whereas the 488 nm curve, which is specific for GFP shows no significant flow through of GFP which indicates a selective binding of GFP-(HR)₄. During the loading, the 488 nm curve rises slowly indicating the flow through of GFP-(RH)₄ which also was observed by a light green coloring of the flow through by eye. The increasing concentration of GFP-RH4 in the flow through can also be seen on the SDS gel from lane 1 to lane 6 as the band of GFP-(RH)4 is more pronounced After loading the column is equilibrated to WB1. During this equilibration, the 280 nm curve falls back to baseline level. The 488 nm curve falls significant slower back to baseline level which indicates washing out of GFP-RH4 of the column. This can also be seen on the SDS gel in lanes 7 and 9 where mostly GFP-(RH)₄ is seen. Following this a washing step with WB2 containing a high salt concentration (1 M NaCl) was performed to wash out proteins bound by weak ion interactions to the column. Both the 288 nm and 488 nm curves show no significant elution of any protein. After the washing step the elution with E1 buffer over 20 CV is initiated. The GFP-(RH)₄ elutes at a lysine concentration of about 150 mM. The eluting peak (10 mL) is collected in four fractions: The start of the elution (1 mL), the front part of the peak (1 mL), the peak part of the elution (4 mL) and the tail of the elution peak (4 mL). The elution fractions can be seen in the lanes 10 to 13 on the SDS gel. In all four lanes a clear GFP-(RH)₄ band can be seen. The purity of every lane was calculated with the Amersham Typhoon and resulted in purities for lane 10 to 13 of 82 %, 78 %, 86 % and 94% respectively. In all four fractions the main contaminates are two proteins with a size if about 30 kDa and 43 kDa respectively. The protein at 43 kDa is likely to be an RNA binding Protein as also found in similar approaches.

### EXAMPLE 5 - AFFINITY PROTEIN PURIFICATION WITH UNDERIVATIZED SILICA COLUMNS

### Summary

Example 5 shows that the novel tag system as provided herein (GFP-(RH)₄) can be used for affinity protein purification with underivatized silica columns. This is a significant improvement to existing separation affinity-based methods such as immobilized metal affinity columns (IMAC), where hazardous materials such as imidazole are needed for protein elution.

In Example 5 protein purities of up to 85% directly from crude cell lysates (tested with PosH-Tag fused to GFP) are achieved with one binding step, one washing step and one elution step in a silica column (75 mL). Therefore, this purity is reached with just one capture step. Other proteins, such as the enzyme enereductase and a polymerized B domain from protein A, can be purified by the same method as well. However, there is still optimization necessary and possible. We already observed higher purities of 90-95% for tagged proteins when the column is overloaded which is even closer to realistic processing of the protein purification.

### Immobilization

If a strong acid such as HCl (0.1 M) is added, the bond is irreversible, and the proteins cannot be eluted with lysine anymore. However, with boiling in SDS, an elution of denatured proteins is possible.

### Experimental Details

For cleared lysate purification a XK16 column (GE Healthcare, Germany) filled with Davisil 643 (Sigma, Germany) was prepared with a column volume (CV) of 75 mL. The column was equilibrated with 4 CV of equilibration buffer (50 mM Tris pH 8.0) at a flowrate of 4 mL min⁻¹. The equilibration was followed by loading of the cleared lysate on the column. Once the lysate was loaded on the column, it was washed with 4 CV of equilibration buffer. As soon as the UV signal decreased back to its baseline, the elution process was started. Isocratic elution was performed with a 50 mM Tris pH 8.0 + 500 mM L-lysine buffer.

For the static binding capacity, the purified GFP was loaded on silicas with different pore sizes:

| Silica | Particle size [µm] | Pore size [Å] | Specific surface area [m² g⁻¹] |
|---|---|---|---|
| Silica gel 60 | 40-63 | 60 | 500 |
| Davisil 643 | 35-70 | 150 | 300 |
| SiliCycle 500 | 40-75 | 500 | 47 |

A concentration of 1 g L⁻¹ silica per tube was used at a total volume of 1 mL. The tubes were placed in a table-top shaker at 750 rpm, 10° C. overnight. The tubes were prepared in technical triplicate. To obtain the amount of protein bound to the silica particles a supernatant analysis BCA and particle BCA was conducted.

For the immobilization experiments purified 1 g L⁻¹ GFP-(RH)₄ was bound to 1 g L⁻¹ silica (Davisil) while in 50 mM Tris pH 8.0. After 1 h incubation the supernatant was discarded and 1 mL of 50 mM Tris with a pH of 2, 3, 4, 5, or 6 was added to the tube. After 1 h incubation the supernatant was discarded again, and the silica was washed 3x with 50 mM Tris pH 8. After washing, an elution step with 50 mM Tris pH 9.0 + 500 mM L-lysine was performed. After washing again with 50 mM Tris pH 8.0 buffer the silica was loaded on a SDS gel to check for remaining protein.

### Results

The transferability of the (RH)₄ tag to other proteins was successfully implemented. The successful purification of (HR)₄-ER and B8-(RH)₄ shows that the (RH)₄ tag is functional on both, the C- and N-terminus. Purities of 80-85%, obtained from densitometry of SDS PAGEs was lower, can be attributed to the fact that in these runs no overloading but upscaling was performed (FIG. 13). When overloading the column with protein of interest purities >95% can be expected. Combining this step with another chromatographic step such as an ion exchange chromatography or immobilized metal afinity chromatography is a promising outlook for upscaling the process.

The adsorption behavior is similar for all investigated silica species (FIG. 13). However, silica with an average pore sizes of 6 nm and of 50 nm demonstrate a lower binding capacity for tagged GFP. This can be explained by the small and large pore sizes, respectively. Furthermore, the Silica 500 has a smaller specific surface area. The affinity of the tag to the silica surfaces is very similar for 15 and 50 nm silica pore sizes with 0.45 and 0.32 g L⁻¹, respectively. This verifies the high affinity of the tag to silica surfaces. The lower affinity to silica with 6 nm pores (1.1 g L⁻¹) can be explained by mass transport limitations into the pores. This pore size is too small for protein purification of an enzyme such as ene reductase.

Aside from purification using the “novel peptide tag”, we found out that an immobilization on silica is possible, as well. If a tagged protein (e.g. GFP) is loaded on silica particles (such as chromatography resin), the tagged protein binds to the surface at typical buffer conditions (TRIS 50 mM pH 8). As we tested different possibilities for protein elution, we found out that a low pH such as pH 2 or 3 does not lead to elution of the protein. While tagged protein incubated with pH 4-6 can be removed with the elution buffer (500 mM lysine and 50 mM Tris pH 9.0), the proteins incubated at pH 2 and 3 cannot be eluted with the elution buffer. We verified the adsorption with on-particle SDS PAGE (FIG. 15). Hence, the tagged proteins seem to be separable from silica when heated up to 90° C. in an SDS containing solution.

The results shown above illustrate the possibility to immobilize proteins with the help of the novel tag as described herein on silica, in particular on underivatized silica.

### REFERENCES

Berensmeier, S.; Blank-Shim, S.; Schwaminger, S.; Fraga-Garcia, P.; Wenzel, W.; Borkowska-Panek, M.; Anand, P.; Fink, K. Magnetic particle-binding peptides.

Terpe, K. Overview of tag protein fusions: From molecular and biochemical fundamentals to commercial systems. Applied microbiology and biotechnology 2003, 60, 523-533.

Colombo, M.; Carregal-Romero, S.; Casula, M. F.; Gutiérrez, L.; Morales, M. P.; Böhm, I. B.; Heverhagen, J. T.; Prosperi, D.; Parak, W. J. Biological applications of magnetic nanoparticles. Chemical Society reviews 2012, 41, 4306-4334.

Le Sage, D.; Arai, K.; Glenn, D. R.; DeVience, S. J.; Pham, L. M.; Rahn-Lee, L.; Lukin, M. D.; Yacoby, A.; Komeili, A.; Walsworth, R. L. Optical magnetic imaging of living cells. Nature 2013, 496, 486-489.

Laurent, S.; Forge, D.; Port, M.; Roch, A.; Robic, C.; Vander Elst, L.; Muller, R. N. Magnetic iron oxide nanoparticles: Synthesis, stabilization, vectorization, physicochemical characterizations, and biological applications. Chemical reviews 2008, 108, 2064-2110.

Berensmeier, S. Magnetic particles for the separation and purification of nucleic acids. Applied microbiology and biotechnology 2006, 73, 495-504.

Schwaminger, S. P.; Bauer, D.; Fraga-García, P.; Wagner, F. E.; Berensmeier, S. Oxidation of magnetite nanoparticles: Impact on surface and crystal properties. CrystEngComm 2017, 19, 246-255.

Roth, H.-C.; Schwaminger, S. P.; Schindler, M.; Wagner, F. E.; Berensmeier, S. Influencing factors in the CO-precipitation process of superparamagnetic iron oxide nano particles: A model based study. Journal of Magnetism and Magnetic Materials 2015, 377, 81-89.

Massart, R. Preparation of aqueous magnetic liquids in alkaline and acidic media. IEEE Trans. Magn. 1981, 17, 1247-1248.

Kuroda, A.; Ikeda, T.; Funabashi, H. Purification method, purification kit, and silicon oxide-binding tag for use therein.

Baneyx, F. C.; Coyle, B. L. Affinity tags and processes for purifying and immobilizing proteins using same.

Schwaminger, S. P.; Fraga-García, P.; Eigenfeld, M.; Becker, T. M.; Berensmeier, S. Magnetic Separation in Bioprocessing Beyond the Analytical Scale: From Biotechnology to the Food Industry. Frontiers in bioengineering and biotechnology 2019, 7, 233.

Block, H.; Maertens, B.; Spriestersbach, A.; Brinker, N.; Kubicek, J.; Fabis, R.; Labahn, J.; Schäfer, F. Chapter 27 Immobilized-Metal Affinity Chromatography (IMAC). Guide to Protein Purification, 2nd Edition; Methods in Enzymology; Elsevier, 2009; pp 439-473.

Hearn, M. T.; Acosta, D. Applications of novel affinity cassette methods: Use of peptide fusion handles for the purification of recombinant proteins. Journal of molecular recognition : JMR 2001, 14, 323-369.

Gaberc-Porekar, V.; Menart, V. Potential for Using Histidine Tags in Purification of Proteins at Large Scale. Chem. Eng. Technol. 2005, 28, 1306-1314.

Franzreb, M.; Siemann-Herzberg, M.; Hobley, T. J.; Thomas, O. R. T. Protein purification using magnetic adsorbent particles. Applied microbiology and biotechnology 2006, 70, 505-516.

Uzun, K.; Çevik, E.; Senel, M.; Sözeri, H.; Baykal, A.; Abasiyanik, M. F.; Toprak, M. S. Covalent immobilization of invertase on PAMAM-dendrimer modified superparamagnetic iron oxide nanoparticles. J Nanopart Res 2010, 12, 3057-3067.

Johnson, A. K.; Zawadzka, A. M.; Deobald, L. A.; Crawford, R. L.; Paszczynski, A. J. Novel method for immobilization of enzymes to magnetic nanoparticles. J Nanopart Res 2008, 10, 1009-1025.

Chang, C.-C.; Lee, S.-F.; Sun, K.-W.; Ho, C.-C.; Chen, Y.-T.; Chang, C.-H.; Kan, L.-S. Mn,Cd-metallothionein-2: A room temperature magnetic protein. Biochemical and biophysical research communications 2006, 340, 1134-1138.

Baalousha, M. Aggregation and disaggregation of iron oxide nanoparticles: Influence of particle concentration, pH and natural organic matter. The Science of the total environment 2009, 407, 2093-2101.

Schnell, F.; Kube, M.; Berensmeier, S.; Schwaminger, S. P. Magnetic Recovery of Cellulase from Cellulose Substrates with Bare Iron Oxide Nanoparticles. ChemNanoMat 2019, 5, 422-426.

Roth, H.-C.; Schwaminger, S. P.; Peng, F.; Berensmeier, S. Immobilization of Cellulase on Magnetic Nanocarriers. ChemistryOpen 2016, 5, 183-187.

Shaw, S.-Y.; Chen, Y.-J.; Ou, J.-J.; Ho, L. Preparation and characterization of Pseudomonas putida esterase immobilized on magnetic nanoparticles. Enzyme and Microbial Technology 2006, 39, 1089-1095.

Zhang, X.; Soori, G.; Dobleman, T. J.; Xiao, G. G. The application of monoclonal antibodies in cancer diagnosis. Expert review of molecular diagnostics 2014, 14, 97-106.

Saeed, A. F. U. H.; Wang, R.; Ling, S.; Wang, S. Antibody Engineering for Pursuing a Healthier Future. Frontiers in microbiology 2017, 8, 495.

Kelley, B. Very large scale monoclonal antibody purification: The case for conventional unit operations. Biotechnology progress 2007, 23, 995-1008.

Process scale purification of antibodies; Gottschalk, U., Ed., Second edition; John Wiley & Sons Inc: Hoboken, NJ, 2017.

Shukla, A. A.; Hubbard, B.; Tressel, T.; Guhan, S.; Low, D. Downstream processing of monoclonal antibodies--application of platform approaches. Journal of chromatography. B, Analytical technologies in the biomedical and life sciences 2007, 848, 28-39.

Carta, G.; Jungbauer, A. Protein chromatography: Process development and scale-up; Wiley-VCH: Weinheim, 2010.

Schwaminger, S. P.; Blank-Shim, S. A.; Scheifele, I.; Fraga-García, P.; Berensmeier, S. Peptide binding to metal oxide nanoparticles. Faraday discussions 2017, 204, 233-250.

Freiherr von Roman, M.; Berensmeier, S. Improving the binding capacities of protein A chromatographic materials by means of ligand polymerization. Journal of chromatography. A 2014, 1347, 80-86.

Sanchayita Ghose, Thomas M. McNerney, Brian Hubbard, Preparative protein purification on underivatized silica, 2004, 87(3):413-423, BIOTECHNOLOGY AND BIOENGINEERING,

1. A peptide consisting of a sequence of 5 or 6 to 30, preferably 6 to 12, most preferably 10 to 12 amino acids, wherein
(a) at least ⅓, preferably at least ½ of said amino acids are amino acids having a functional group or a side chain which is positively charged at neutral pH; and
(b) at least two amino acids, preferably at least three are histidines.

2. The peptide of claim 1, wherein the amino acids having a functional group or a side chain which is positively charged at neutral pH are lysines, arginines or a mixture of lysine(s) and arginine(s).

3. The peptide of claim 1, wherein the peptide comprises at least one, preferably at least two and most preferably at least three alternating region(s) (S) and regions (W) each, wherein
each region (S) comprises at least one amino acid having a functional group or a side chain which is positively charged at neutral pH, and
each region (W) comprises at least one histidine.

4. The peptide of claim 1 wherein the at least two, preferably at least three histidines are present in a stretch or no more than 8 consecutive amino acids, preferably not more than 6 consecutive amino acids, and most preferably not more than 4 consecutive amino acids.

5. The peptide of claim 1, wherein any amino acids present in the peptide and having no functional group or side chain or having a functional group or side chain which is negatively charged at neutral pH are present in the peptide up to ⅕, preferably up to ⅒ and most preferably up to 1/20 of all amino acids of the peptide, and wherein said amino acids preferably
(i) do have a functional group or a side chain which is negatively charged at neutral pH, and/or
(ii) have a functional group or a side chain which does not bear a net charge at neutral pH.

6. A (poly)peptide being tagged with the peptide according to claim 1 .

7. The (poly)peptide of claim 6, wherein the peptide is genetically fused or chemically linked to the (poly)peptide.

8. The (poly)peptide of claim 6, wherein the (poly)peptide is an antibody or an antibody fragment, protein A, a hormone, an enzyme, a binding protein, a receptor, a ligand or a pharmaceutically active_(poly)peptide.

9. An inorganic surface comprising at least one peptide according to claim 1 and/or at least one (poly)peptide being tagged with the peptide, wherein the peptide and/or the (poly)peptide is directly bound to the inorganic surface.

10. The inorganic surface of claim 9, wherein the inorganic surface comprises iron oxide or silica.

11. The inorganic surface of claim 9, wherein the inorganic surface is a particle, preferably a nanoparticle or a microparticle.

12. The inorganic surface of claim 10, wherein
(i) the iron oxide comprises ferrous oxide, magnetite, maghemite or a transition state between magnetite and maghemite, and/or
(ii) the silica comprises silanol or siloxane.

13. A microorganism or a host cell expressing the peptide according to claim 1 and/or a (poly)peptide being tagged with the peptide .

14. A method for immobilizing the peptide according to claim 1 and/or a (poly)peptide being tagged with the peptide on an inorganic surface, the method comprising
contacting the inorganic surface with the peptide, the (poly)peptide or a microorganism or host cell under conditions,
wherein the microorganism or host cell expresses the peptide or the (poly)peptide, and
wherein the peptide and/or the (poly)peptide directly binds to the inorganic surface via the peptide .

15. A method for purifying the peptide according to claim 1 and/or a (poly)peptide being tagged with the peptide, the method comprising
(a) contacting an inorganic surface with a mixture comprising the peptide, the (poly)peptide and/or a microorganism or host cell under conditions, wherein the microorganism or host cell expresses the peptide or the (poly)peptide, and wherein the peptide and/or the (poly)peptide directly binds to the inorganic surface via the peptide, and
(b) optionally eluting the peptide and/or the (poly)peptide from the inorganic surface, thereby purifying the peptide and/or the (poly)peptide from said mixture.


SEQUENCE LISTING (11 sequences)

SEQ ID NO: 1 - protein, 10 aa, Artificial Sequence
  peptide
  VARIANT 9: E or D
HRHRHRHRXR

SEQ ID NO: 2 - protein, 10 aa, Artificial Sequence
  peptide
  VARIANT 5,10: E or D
HRHRXHRHRX

SEQ ID NO: 3 - protein, 10 aa, Artificial Sequence
  peptide
  VARIANT 1: E or D
XHHRRHHRRH

SEQ ID NO: 4 - protein, 10 aa, Artificial Sequence
  peptide
  VARIANT 5: E or D
HHRRXHHRRH

SEQ ID NO: 5 - protein, 10 aa, Artificial Sequence
  peptide
  VARIANT 5,10: E or D
HRHRXHRHRX

SEQ ID NO: 6 - protein, 10 aa, Artificial Sequence
  peptide
  VARIANT 1: E or D
XHHRRHHRRH

SEQ ID NO: 7 - protein, 10 aa, Artificial Sequence
  peptide
  VARIANT 5: E or D
HHRRXHHRRH

SEQ ID NO: 8 - protein, 10 aa, Artificial Sequence
  peptide
  VARIANT 9: A,C,F,G,I,L,M,N,P,Q,S,T,V or W
  VARIANT 10: E or D
HRHRHRHRYX

SEQ ID NO: 9 - protein, 10 aa, Artificial Sequence
  peptide
  VARIANT 5: E or D
  VARIANT 10: A,C,F,G,I,L,M,N,P,Q,S,T,V or W
HRHRXHRHRY

SEQ ID NO: 10 - protein, 10 aa, Artificial Sequence
  peptide
  VARIANT 1: E or D
  VARIANT 8: A,C,F,G,I,L,M,N,P,Q,S,T,V or W
XHHRRHHYRH

SEQ ID NO: 11 - protein, 10 aa, Artificial Sequence
  peptide
  VARIANT 5: A,C,F,G,I,L,M,N,P,Q,S,T,V or W
  VARIANT 9: E or D
HHRRYHHRXH